(19) United States
(12) Patent Application Publication
(10) Pub. No.: US 2021/0272680 A1
(43) Pub. Date: Sep. 2, 2021

(54) METHOD, APPARATUS AND DEVICE FOR CONVERTING DICOM BASED FILE

(71) Applicant: BOE TECHNOLOGY GROUP CO., LTD., Beijing (CN)

(72) Inventor: Lei SHAO, Beijing (CN)

(73) Assignee: BOE TECHNOLOGY GROUP CO., LTD., Beijing (CN)

(21) Appl. No.: 17/255,690

(22) PCT Filed: May 27, 2020
(86) PCT No.: PCT/CN2020/092534

§ 371 Date: Dec. 23, 2020

(30) Foreign Application Priority Data
May 30, 2019 (CN) 201910463298.2

(51) Int. Cl. G16H 30/40; G16H 30/20; G06F 16/11

(52) U.S. Cl. CPC G16H 30/40; G06F 16/116; G16H 30/20

(57) ABSTRACT

A method, apparatus and device for converting a DICOM based file are provided. The method includes: acquiring a DICOM tag of a DICOM file to be converted, the DICOM tag including a tag description and a tag value; and converting the DICOM file to be converted into a JSON format file according to the DICOM tag.

### CROSS-REFERENCE TO RELATED APPLICATION

The present application claims a priority to Chinese Patent Application No. 201910463298.2 filed in China on May 30, 2019, a disclosure of which is incorporated in its entirety by reference herein.

### TECHNICAL FIELD

The present disclosure relates to the field of file processing technology, in particular to a method, apparatus and device for converting a digital imaging and communications in medicine (DICOM) based file.

### BACKGROUND

DICOM is an international standard for medical image information and defines a medical image format that provides quality satisfying a clinical requirement and can be used for data interchange. DICOM is widely applied to radiotherapy equipment, cardiovascular imaging equipment, and radiological diagnosis and treatment equipment (X-ray, CT, nuclear magnetic resonance, and ultrasound), and have found increasingly intensive and extensive applications in ophthalmology, dentistry, among other medical fields.

A DICOM file complies with a DICOM file storage format and is used for archiving DICOM file information generated from examining a patient. A DICOM file is equivalent to a file-level database and is used to store patient information, patient study information, study series information, and study image information, but does not include pixel information of a DICOM image.

For a DICOM file that meets a DICOMDIR standard and the analysis of DICOMDIR content, the file has to be analyzed by a professional software before the content of the file can be viewed. The DICOM file is suitable for use by professional users, and ordinary users need to be familiar with the use of the professional DICOMDIR analysis software to view DICOMDIR content.

### SUMMARY

The present disclosure provides in an embodiment a method for converting a DICOM based file, including:

acquiring a DICOM tag of a DICOM file to be converted, the DICOM tag including a tag description and a tag value; and

converting the DICOM file to be converted into a JavaScript object notation (JSON) format file according to the DICOM tag.

Optionally, the tag value includes a single value, a plurality of values, or a null value.

Optionally, a data structure of the DICOM file to be converted has a plurality of levels, and data at each level has the DICOM tag.

Optionally, a data structure of the DICOM file to be converted has at least one of following levels: a patient level, a study level, a series level, an image level.

Optionally, in the method, the acquiring the DICOM tag of the DICOM file to be converted includes:

acquiring the DICOM tag of the data at each level in the DICOM file to be converted; and

the converting the DICOM file to be converted into the JSON format file according to the DICOM tag includes:

determining a key and a value of a JSON information item corresponding to the data at each level according to the DICOM tag of the data at the each level; and

acquiring the JSON format file according to the key and the value of the JSON information item corresponding to the data at each level.

Optionally, in the method, the determining the key and the value of the JSON information item corresponding to the data at each level according to the DICOM tag of the data at the each level in the JSON format file obtained through conversion includes:

determining a key of a JSON information item corresponding to data at a target level among the plurality of levels according to the tag description corresponding to the DICOM tag of the data at the target level; and determining a value of the JSON information item corresponding to the data at the target level according to the tag value corresponding to the DICOM tag of the data at the target level.

Optionally, in the method, the determining the key of the JSON information item corresponding to the data at the target level among the plurality of levels according to the tag description corresponding to the DICOM tag of the data at the target level includes:

using the tag description corresponding to the DICOM tag of the data at the target level as the key of the JSON information item corresponding to the data at the target level.

Optionally, in the method, the determining the value of the JSON information item corresponding to the data at the target level according to the tag value corresponding to the DICOM tag of the data at the target level includes:

in case that the tag value corresponding to the DICOM tag of the data at the target level is a single value, using the tag value of the DICOM tag as the value of the JSON information item corresponding to the data at the target level;

in case that the tag value corresponding to the DICOM tag of the data at the target level is a plurality of values, converting the plurality of values of the DICOM tag into a JSON format array object, and using the JSON format array object as the value of the JSON information item corresponding to the data at the target level; and

in case that the tag value corresponding to the DICOM tag of the data at the target level is a null value, using the null value NULL as the value of the JSON information item in the converted JSON format file corresponding to the data at the target level.

Optionally, the method further includes:

sequentially using the data at the levels as the data at the target level according to a loop algorithm, wherein data at the study level is nested in data at the patient level, data at the series level is nested in data at the study level, data at the image level is nested in data at the series level, and data at an upper level includes at least one group of data at a lower level.

The present disclosure further provides in an embodiment an apparatus for converting a DICOM based file, including:

an acquisition module, configured to acquire a DICOM tag of a DICOM file to be converted, the DICOM tag including a tag description and a tag value;

a conversion module, configured to convert the DICOM file to be converted into a JSON format file according to the DICOM tag.

Optionally, in the apparatus, the acquisition module is specifically configured to:

acquire the DICOM tag of the data at each level in the DICOM file to be converted;

the conversion module includes:

an information determination unit, configured to determine a key and a value of a JSON information item corresponding to the data at each level according to the DICOM tag of the data at the each level;

a file acquisition unit, configured to acquire the JSON format file according to the key and the value of the JSON information item corresponding to the data at each level.

Optionally, in the apparatus, the information determination unit is specifically configured to:

determine a key of a JSON information item corresponding to data at a target level among the plurality levels according to the tag description corresponding to the DICOM tag of the data at the target level; and determine a value of the JSON information item corresponding to the data at the target level according to the tag value corresponding to the DICOM tag of the data at the target level.

Optionally, in the apparatus, to determine the key of the JSON information item corresponding to the data at the target level among the plurality of levels according to the tag description corresponding to the DICOM tag of the data at the target level, the information determination unit is specifically configured to:

use the tag description corresponding to the DICOM tag of the data at the target level as the key of the JSON information item corresponding to the data at the target level.

Optionally, in the apparatus, to determine the value of the JSON information item corresponding to the data at the target level according to the tag value corresponding to the DICOM tag of the data at the target level, the information determination unit is specifically configured to:

in case that the tag value corresponding to the DICOM tag of the data at the target level is a single value, use the tag value of the DICOM tag as the value of the JSON information item corresponding to the data at the target level;

in case that the tag value corresponding to the DICOM tag of the data at the target level is a plurality of values, convert a plurality of tag values of the DICOM tag into a JSON format array object, and use the JSON format array object as the value of the JSON information item corresponding to the data at the target level;

in case that the tag value corresponding to the DICOM tag of the data at the target level is a null value, use the null value NULL as the value of the JSON information item in the converted JSON format file corresponding to the data at the target level.

Optionally, the apparatus further includes: a target level data determination unit, configured to sequentially use the data at the levels as the data at the target level according to a loop algorithm, wherein data at the study level is nested in data at the patient level, data at the series level is nested in data at the study level, data at the image level is nested in data at the series level, and data at an upper level includes at least one group of data at a lower level.

The present disclosure further provides in an embodiment a device for converting a DICOM based file, including a storage, a processor, and a computer program stored in the storage and configured to be executed by the processor, wherein the processor is configured to execute the computer program to implement the foregoing method.

### BRIEF DESCRIPTION OF THE DRAWINGS

FIG. 1 is schematic flowchart of an embodiment of a conversion method according to an embodiment of the present disclosure;

FIG. 2 is a schematic flowchart of step S120 in FIG. 1;

FIG. 3 is a schematic flowchart of a specific implementation using the conversion method according to an embodiment of the present disclosure;

FIG. 4 is a schematic structural diagram of a conversion apparatus according to an embodiment of the present disclosure.

### DETAILED DESCRIPTION

To make the technical problems, the technical solutions, and advantages of the present disclosure clearer, detailed descriptions are provided below with reference to the accompanying drawings and specific embodiments.

To achieve the content analysis of a conventional DICOM file, professional software needs to be used, and as a result, there is no versatile means for viewing a DICOM file. The present disclosure provides in an embodiment a method for converting a DICOM file, which is capable of converting a DICOM file into a text file in a JSON format. The content of the text file in the JSON format can be viewed by using a TXT view tool or the like in a simple and easy manner, such that a versatile means for viewing the DICOM file is provided, thereby solving the problem that it is inconvenient to view the content of the DICOM file.

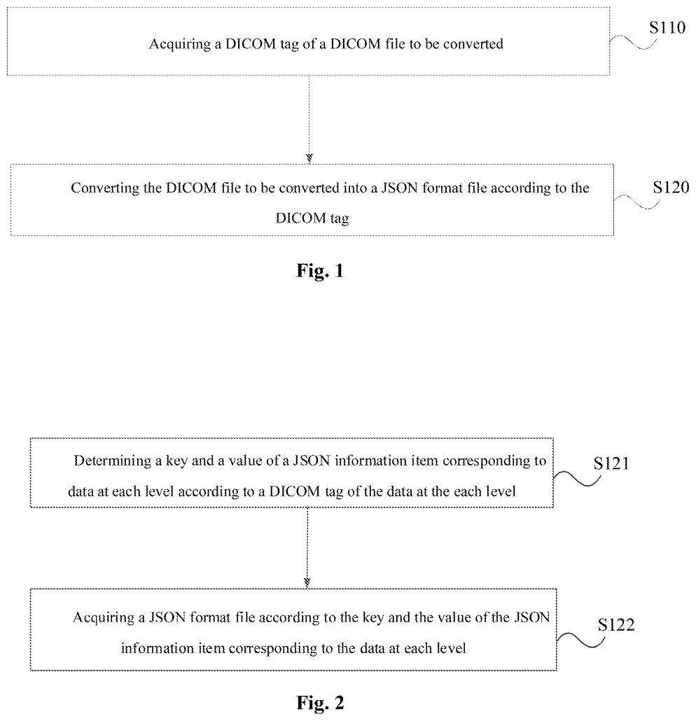

Specifically, referring to FIG. 1, a method for converting a DICOM file according to an embodiment of the present disclosure includes the following steps:

S110: Acquiring a DICOM tag of a DICOM file to be converted, the DICOM tag including a tag description and a tag value, the tag value including a single value, a plurality of values, or a null value;

S120: Converting the DICOM file to be converted into a JSON format file according to the DICOM tag.

In the method for converting a DICOM file according to the embodiment of the present disclosure, a DICOM file to be converted can be converted into a JSON format file according to a DICOM tag of the DICOM file to be converted, so that a versatile means for viewing the DICOM file is provided, thereby solving the problem that it is inconvenient to view the content of the DICOM file.

JSON is a lightweight data-interchange format, is based on a subset of ECMAScript (a JavaScript standard standardized by European Computer Manufacturers Association (ECMA)), and stores and represents data in a text format completely independent of a programming language. With a simple and clear hierarchical structure, JSON becomes an ideal data-interchange language, which is easy to read and write, is easy for machines to parse and generate, and can effectively improve the efficiency of network transmission.

Therefore, by converting a DICOM file to be converted into a JSON format file, the viewing of the DICOM file can be simpler and more convenient.

Specifically, in step S110, the acquiring the DICOM tag of the DICOM file to be converted includes:

acquiring the DICOM tag of the data at each level in the DICOM file to be converted.

In step S120, the converting the DICOM file to be converted into a JSON format file according to the DICOM tag, as shown in FIG. 2, includes:

S121: Determining the key and the value of the JSON information item in the converted JSON format file corresponding to the data at each level according to the DICOM tag of the data at the each level;

S122: Acquiring the JSON format file according to the key and the value of the JSON information item corresponding to the data at each level.

It needs to be noted that each DICOM file carries a variety of information. The information may be specifically categorized into data information at four levels. Content of data at the first level corresponds to patient information, content of data at the second level corresponds to study information, content of data at the third level corresponds to series information, and content of data at the fourth level corresponds to image information.

Information at the patient level is at the top level. One piece of information at the patient level may include a plurality pieces of information at the study level. Each piece of information at the study level may include a plurality pieces of information at the series level. Each piece of information at the series level may include a plurality pieces of information at the image level. The information at the image level is a part in the DICOM file related to image information, and includes an actual storage path of the DICOM file on a magnetic disk, but does not include image pixel information of the DICOM file.

The information at each level may be identified by a DICOM tag. The DICOM tag is formed by two hexadecimal numbers, i.e., (group, element). For example, (0010, 0010) may be used to represent a patient's name.

Specifically, the information at each level is encapsulated into a fundamental unit, that is, a data element. Each data element is formed by four parts:

DICOM tag, used for storing an identifier of information at the level;

Value representation (VR), used for storing a data type of the information at the level;

Value length (data length), used for storing a data length describing the information at the level;

Value Field (data field), used for storing a data value describing the information at the level.

Specifically, a data type of the DICOM file stored in the VR part includes: AE, AS, AT, CS, DA, DS, DT, FD, IS, LO, LT, PN, SH, SL, SQ, SS, ST, TM, UC, UI, UL, UN, UR, US, UT, and the like. The data type of the DICOM file stored in the VR part being SQ represents that the value Field part stores one or more nested data values.

In the conversion method in the embodiment of the present disclosure, a DICOM tag of data at each level in a DICOM file to be converted is acquired, and a key and a value of a JSON information item corresponding to the data at each level are determined according to the DICOM tag of the data at each level. A JSON format file is acquired according to the key and the value of the JSON information item corresponding to the data at each level.

In the embodiment of the present disclosure, specifically, in step S121, the determining the key and the value of the JSON information item corresponding to the data at each level according to the DICOM tag of the data at the each level includes: determining a key of a JSON information item corresponding to data at a target level among the plurality of levels according to the tag description corresponding to the DICOM tag of the data at the target level; and determining a value of the JSON information item corresponding to the data at the target level according to the tag value corresponding to the DICOM tag of the data at the target level.

It needs to be noted that in a DICOM file, each data element is uniquely determined and distinguished from another data element by a DICOM tag in the data element. In addition, correspondingly, each data element uniquely determined by the DICOM tag includes a corresponding tag description. The tag description is represented in a character string form and used to record description information of the data at the target level. By converting the tag description corresponding to the DICOM tag into a key of a JSON information item in the JSON format file, the description information corresponding to the data at each level can be converted into the key of the JSON information item in the JSON format file, to be recorded in the JSON format file. Optionally, a data type of the key of the JSON information item is a character string.

In addition, the tag value corresponding to the DICOM tag of the data at the target level is a value stored in the value Field in the data element, and a tag value corresponding to the data at each level can be converted according to the DICOM tag into the value of the JSON information item in the JSON format file, so that the value stored in the value Field corresponding to the data at each level can be recorded in the JSON format file.

Specifically, in case that the data type in the VR recorded in the data element of the data at the target level varies, a data type of the key of the converted JSON information item varies correspondingly according to the tag value of the DICOM tag of the data at the target level. A specific correspondence may be determined according to the following Table 1.

TABLE 1

| DICOM data type | JSON data type |
|---|---|
| AE | String |
| AS | String |
| AT | String |
| CS | String |
| DA | String |
| DS | Number |
| DT | String |
| FL | Number |
| FD | Number |
| IS | Number |
| LO | String |
| LT | String |
| PN | String |
| SH | String |
| SL | Number |
| SQ | Array of JSON Objects |
| SS | Number |
| ST | String |
| TM | String |
| UC | String |
| UI | String |
| UL | Number |
| UN | String |
| UR | String |
| US | Number |
| UT | String |

In the foregoing manner, the data at the levels are sequentially used as the data at the target level, a key of a JSON information item in the JSON format file corresponding to the data at the target level is determined according to the tag description corresponding to the DICOM tag of the data at the target level; and a value of the JSON information item in the JSON format file corresponding to the data at the target level is determined according to the tag value corresponding to the DICOM tag of the data at the target level.

In this way, the key and the value of the JSON information item corresponding to the data at each level may be sequentially acquired, to acquire the JSON format file.

Specifically, the determining the key of the JSON information item corresponding to the data at the target level among the plurality of levels according to the tag description corresponding to the DICOM tag of the data at the target level includes:

using the tag description corresponding to the DICOM tag of the data at the target level as the key of the JSON information item corresponding to the data at the target level.

Specifically, the determining the value of the JSON information item corresponding to the data at the target level according to the tag value corresponding to the DICOM tag of the data at the target level includes:

in case that the tag value corresponding to the DICOM tag of the data at the target level is a single value, using the tag value of the DICOM tag as the value of the JSON information item corresponding to the data at the target level;

in case that the tag value corresponding to the DICOM tag of the data at the target level is a plurality of values, converting the plurality of values of the DICOM tag into a JSON format array object, and using the JSON format array object as the value of the JSON information item corresponding to the data at the target level;

in case that the tag value corresponding to the DICOM tag of the data at the target level is a null value, using the null value NULL as the value of the JSON information item corresponding to the data at the target level.

In the foregoing manner, the key and the value of the JSON information item corresponding to the data at each level can be determined according to the DICOM tag of the data at the each level. On this basis, according to a key and a value of a JSON information item corresponding to data at the first level, the converted JSON format file can be acquired.

Optionally, in the method for converting a DICOM file according to the embodiment of the present disclosure, in step S121, the determining the key and the value of the JSON information item corresponding to the data at each level according to the DICOM tag of the data at the each level includes:

sequentially using the data at the levels as the data at the target level according to a loop algorithm and according to an arrangement sequence of data at the following levels in the DICOM file to be converted, wherein, data at the study level is nested in data at the patient level, data at the series level is nested in the data at the study level, data at the image level is nested in the data at the series level, and data at an upper level including at least one group of data at a lower level; determining the key and the value of the JSON information item corresponding to the data at the target level according to the DICOM tag of the data at the target level:

the data at the patient level;

the data at the patient level includes a plurality of objects at the study level, the plurality of objects at the study level form an array set, and each object at the study level may be used as an object at the study level of a target item;

the object at the study level of the target item includes a plurality of objects at the series level, the plurality of objects at the series level form an array set, and each object at the series level may be used as the object at the series level of the target item;

the object at the series level of the target item includes a plurality of objects at the image level.

The key and the value of the JSON information item corresponding to the data at the target level are sequentially determined by using the data as the data at the target level according to the foregoing sequence, so that the process of file conversion is sequentially performed, to avoid information omission.

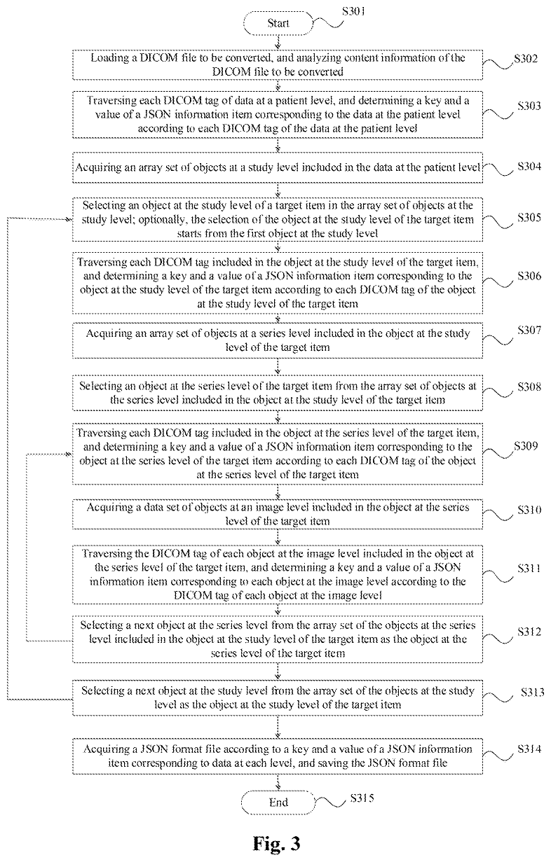

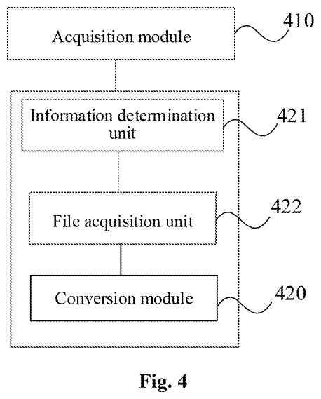

Specifically, referring to FIG. 3, a specific implementation procedure of the method for converting a DICOM file according to an embodiment of the present disclosure may include the following steps:

S301: Start;

S302: Loading a DICOM file to be converted, and analyzing content information of the DICOM file to be converted;

S303: Traversing each DICOM tag of data at a patient level, and determining a key and a value of a JSON information item corresponding to the data at the patient level according to each DICOM tag of the data at the patient level;

S304: Acquiring an array set of objects at a study level included in the data at the patient level;

S305: Selecting an object at the study level of a target item in the array set of objects at the study level; optionally, the selection of the object at the study level of the target item starts from the first object at the study level;

S306: Traversing each DICOM tag included in the object at the study level of the target item, and determining a key and a value of a JSON information item corresponding to the object at the study level of the target item according to each DICOM tag of the object at the study level of the target item;

S307: Acquiring an array set of objects at a series level included in the object at the study level of the target item;

S308: Selecting an object at the series level of the target item from the array set of objects at the series level included in the object at the study level of the target item; optionally, the selection of the object at the series level of the target item starts from the first object at the series level;

S309: Traversing each DICOM tag included in the object at the series level of the target item, and determining a key and a value of a JSON information item corresponding to the object at the series level of the target item according to each DICOM tag of the object at the series level of the target item;

S310: Acquiring a data set of objects at an image level included in the object at the series level of the target item;

S311: Traversing the DICOM tag of each object at the image level included in the object at the series level of the target item, and determining a key and a value of a JSON information item corresponding to each object at the image level according to the DICOM tag of each object at the image level;

S312: Selecting a next object at the series level from the array set of the objects at the series level included in the object at the study level of the target item as the object at the series level of the target item, and returning to perform step S309 until the objects at the series level included in the object at the study level of the target item are all converted;

S313: Selecting a next object at the study level from the array set of the objects at the study level as the object at the study level of the target item, and returning to perform step S305 until each object at the study level included in the objects at the study level is converted;

S314: Acquiring a JSON format file according to a key and a value of a JSON information item corresponding to data at each level, and saving the JSON format file;

S315: End.

The present disclosure further provides in an embodiment an apparatus for converting a DICOM based file. As shown in FIG. 4, the apparatus includes:

an acquisition module 410, configured to acquire a DICOM tag of a DICOM file to be converted, the DICOM tag including a tag description and a tag value; and

a conversion module 420, configured to convert the DICOM file to be converted into a JSON format file according to the DICOM tag.

In the apparatus for converting the DICOM file in the embodiment of the present disclosure, a DICOM file to be converted can be converted into a JSON format file according to a DICOM tag of the DICOM file to be converted, so that a versatile means for viewing the DICOM file is provided, thereby solving the problem that it is inconvenient to view the content of the DICOM file.

Optionally, in the conversion apparatus in the embodiment of the present disclosure, the acquisition module 410 is specifically configured to:

acquire the DICOM tag of the data at each level in the DICOM file to be converted;

the conversion module 420 includes:

an information determination unit 421, configured to determine a key and a value of a JSON information item corresponding to the data at each level according to the DICOM tag of the data at the each level; and

a file acquisition unit 422, configured to acquire the JSON format file according to the key and the value of the JSON information item corresponding to the data at each level.

Optionally, in the conversion apparatus, the information determination unit 421 is specifically configured to:

determine a key of a JSON information item corresponding to data at a target level among the plurality of levels according to the tag description corresponding to the DICOM tag of the data at the target level; and determine a value of the JSON information item corresponding to the data at the target level according to the tag value corresponding to the DICOM tag of the data at the target level.

Optionally, in the conversion apparatus, the determining, by the information determination unit 421, the key of the JSON information item corresponding to the data at the target level among the plurality of levels according to the tag description corresponding to the DICOM tag of the data at the target level specifically includes:

using the tag description corresponding to the DICOM tag of the data at the target level as the key of the JSON information item corresponding to the data at the target level.

Optionally, in the conversion apparatus, the determining, by the information determination unit 421, the value of the JSON information item corresponding to the data at the target level according to the tag value corresponding to the DICOM tag of the data at the target level specifically includes:

in case that the tag value corresponding to the DICOM tag of the data at the target level is a single value, using the tag value of the DICOM tag as the value of the JSON information item corresponding to the data at the target level;

in case that the tag value corresponding to the DICOM tag of the data at the target level is a plurality of values, converting the plurality of values of the DICOM tag into a JSON format array object, and using the JSON format array object as the value of the JSON information item corresponding to the data at the target level;

in case that the tag value corresponding to the DICOM tag of the data at the target level is a null value, using the null value NULL as the value of the JSON information item corresponding to the data at the target level.

Optionally, the conversion apparatus further includes: a target level data determination unit, configured to sequentially use the data at the levels as the data at the target level according to a loop algorithm, data at the study level being nested in data at the patient level, data at the series level being nested in data at the study level, data at the image level being nested in data at the series level, and data at an upper level including at least one group of data at a lower level.

The present disclosure further provides in an embodiment a device for converting a DICOM based file, including a storage, a processor, and a computer program stored in the storage and configured to be executed by the processor, where the processor is configured to execute the computer program to implement the foregoing method.

According to the foregoing detailed description, a person skilled in the art should understand a specific implementation in which a processor of the conversion device executes a computer program to perform the foregoing method. Details are not described herein again.

The foregoing descriptions are optional implementations of the present disclosure. It should be noted that for a person of ordinary skill in the art, several improvements and modifications may further be made without departing from the principle of the present disclosure. These improvements and modifications should also be deemed as falling within the scope of the present disclosure.

1. A method for converting a digital imaging and communications in medicine (DICOM) based file, comprising:
acquiring a DICOM tag of a DICOM file to be converted, the DICOM tag comprising a tag description and a tag value; and
converting the DICOM file to be converted into a JSON format file according to the DICOM tag.

2. The method according to claim 1, wherein the tag value comprises a single value, a plurality of values, or a null value.

3. The method according to claim 1, wherein a data structure of the DICOM file to be converted has a plurality of levels, and data at each level has the DICOM tag.

4. The method according to claim 3, wherein a data structure of the DICOM file to be converted has at least one of following levels: a patient level, a study level, a series level, an image level.

5. The method according to claim 3, wherein
the acquiring the DICOM tag of the DICOM file to be converted comprises: acquiring the DICOM tag of the data at each level in the DICOM file to be converted;
the converting the DICOM file to be converted into the JSON format file according to the DICOM tag comprises:
determining a key and a value of a JSON information item corresponding to the data at each level according to the DICOM tag of the data at the each level;
acquiring the JSON format file according to the key and the value of the JSON information item corresponding to the data at each level.

6. The method according to claim 5, wherein the determining the key and the value of the JSON information item corresponding to the data at each level according to the DICOM tag of the data at the each level comprises:
determining a key of a JSON information item corresponding to data at a target level among the plurality of levels according to the tag description corresponding to the DICOM tag of the data at the target level; and
determining a value of the JSON information item corresponding to the data at the target level according to the tag value corresponding to the DICOM tag of the data at the target level.

7. The method according to claim 6, wherein the determining the key of the JSON information item corresponding to the data at the target level among the plurality of levels according to the tag description corresponding to the DICOM tag of the data at the target level comprises:
using the tag description corresponding to the DICOM tag of the data at the target level as the key of the JSON information item corresponding to the data at the target level.

8. The method according to claim 6, wherein the determining the value of the JSON information item corresponding to the data at the target level according to the tag value corresponding to the DICOM tag of the data at the target level comprises:
in case that the tag value corresponding to the DICOM tag of the data at the target level is a single value, using the tag value of the DICOM tag as the value of the JSON information item corresponding to the data at the target level;
in case that the tag value corresponding to the DICOM tag of the data at the target level is a plurality of values, converting the plurality of values of the DICOM tag into a JSON format array object, and using the JSON format array object as the value of the JSON information item corresponding to the data at the target level;
in case that the tag value corresponding to the DICOM tag of the data at the target level is a null value, using the null value (NULL) as the value of the JSON information item corresponding to the data at the target level.

9. The method according to claim 6, further comprising:
sequentially using the data at the levels as the data at the target level according to a loop algorithm, wherein data at the study level is nested in data at the patient level, data at the series level is nested in data at the study level, data at the image level is nested in data at the series level, and data at an upper level comprises at least one group of data at a lower level.

10-15. (canceled)

16. A device for converting a digital imaging and communications in medicine (DICOM) based file, comprising a storage, a processor, and a computer program stored in the storage and configured to be executed by the processor, wherein the processor is configured to execute the computer program to implement a method for converting the DICOM based file, the method comprises:
acquiring a DICOM tag of a DICOM file to be converted, the DICOM tag comprising a tag description and a tag value; and
converting the DICOM file to be converted into a JSON format file according to the DICOM tag.

17. The device according to claim 16, wherein the tag value comprises a single value, a plurality of values, or a null value.

18. The device according to claim 16, wherein a data structure of the DICOM file to be converted has a plurality of levels, and data at each level has the DICOM tag.

19. The device according to claim 18, wherein a data structure of the DICOM file to be converted has at least one of following levels: a patient level, a study level, a series level, an image level.

20. The device according to claim 18, wherein
the acquiring the DICOM tag of the DICOM file to be converted comprises: acquiring the DICOM tag of the data at each level in the DICOM file to be converted;
the converting the DICOM file to be converted into the JSON format file according to the DICOM tag comprises:
determining a key and a value of a JSON information item corresponding to the data at each level according to the DICOM tag of the data at the each level;
acquiring the JSON format file according to the key and the value of the JSON information item corresponding to the data at each level.

21. The device according to claim 20, wherein the determining the key and the value of the JSON information item corresponding to the data at each level according to the DICOM tag of the data at the each level comprises:
determining a key of a JSON information item corresponding to data at a target level among the plurality of levels according to the tag description corresponding to the DICOM tag of the data at the target level; and
determining a value of the JSON information item corresponding to the data at the target level according to the tag value corresponding to the DICOM tag of the data at the target level.

22. The device according to claim 21, wherein the determining the key of the JSON information item corresponding to the data at the target level among the plurality of levels according to the tag description corresponding to the DICOM tag of the data at the target level comprises:
using the tag description corresponding to the DICOM tag of the data at the target level as the key of the JSON information item corresponding to the data at the target level.

23. The device according to claim 21, wherein the determining the value of the JSON information item corresponding to the data at the target level according to the tag value corresponding to the DICOM tag of the data at the target level comprises:
in case that the tag value corresponding to the DICOM tag of the data at the target level is a single value, using the tag value of the DICOM tag as the value of the JSON information item corresponding to the data at the target level;
in case that the tag value corresponding to the DICOM tag of the data at the target level is a plurality of values, converting the plurality of values of the DICOM tag into a JSON format array object, and using the JSON format array object as the value of the JSON information item corresponding to the data at the target level;
in case that the tag value corresponding to the DICOM tag of the data at the target level is a null value, using the null value (NULL) as the value of the JSON information item corresponding to the data at the target level.

24. The device according to claim 21, wherein the method further comprises:
sequentially using the data at the levels as the data at the target level according to a loop algorithm, wherein data at the study level is nested in data at the patient level, data at the series level is nested in data at the study level, data at the image level is nested in data at the series level, and data at an upper level comprises at least one group of data at a lower level.
